(19) United States
(12) Patent Application Publication
(10) Pub. No.: US 2012/0316387 A1
(43) Pub. Date: Dec. 13, 2012

(54) ADJUSTABLE GASTRIC WRAP (AGW)

(76) Inventor: Monica Ann Volker, Lake Geneva, WI (US)

(21) Appl. No.: 13/492,556

(22) Filed: Jun. 8, 2012

Related U.S. Application Data
(60) Provisional application No. 61/494,595, filed on Jun. 8, 2011.

(51) Int. Cl. A61F 2/02

(52) U.S. Cl. 600/37

(57) ABSTRACT

The invention relates to devices, systems and methods for weight reduction. Specifically it relates to the reduction of the stomach with the use of an adjustable wrap around the stomach that is less invasive compared to other stomach reduction surgeries.

The present application claims priority to U.S. Provisional Patent No. 61/494,595, filed Jun. 8, 2011, the entire disclosure of which is herein incorporated by reference in its entirety.

### FIELD OF INVENTION

The invention relates to devices, systems and methods for weight reduction. Specifically it relates to the reduction of the stomach with the use of an adjustable wrap around the stomach that is less invasive compared to other stomach reduction surgeries.

### BACKGROUND OF THE INVENTION

Bariatric surgery is the term encompassing all of the surgical treatments for morbid obesity, not just gastric bypasses, which make up only one class of such operations.

Bariatric surgery produces weight loss by restricting food intake. There are four different types of stomach reduction surgeries that are typically performed in the United States. These include: Roux-en-Y gastric bypass (RYGB), biliopancreatic diversion with a duodenal switch (BPD-DS), and vertical sleeve gastrectomy (VSG) and the adjustable gastric band (AGB).

According to the American Society of Bariatric Surgery, Bariatric surgery is known to be the most effective and long lasting treatment for morbid obesity and many related conditions, but now mounting evidence suggests it may be among the most effective treatments for metabolic diseases and conditions including type 2 diabetes with remission in 76.8% and significantly improved in 86% of patients, hypertension was eliminated in 61.7% and significantly improved in 78.5% of patients, high cholesterol was reduced in more than 70% of patients, non-alcoholic fatty liver disease, obstructive sleep apnea was eliminated 85.7% of patients and may help with the reduction of other health issues.

As with any surgery each of the four types of bariatric surgeries, as briefly described below, have serious risks and or possible complications.

Roux-en-Y gastric bypass (RYGB) works by restricting food intake and by decreasing the absorption of food. Food intake is limited by a small pouch that is similar in size to the adjustable gastric band. In addition, absorption of food in the digestive tract is reduced by excluding most of the stomach, duodenum, and upper intestine from contact with food by routing food directly from the pouch into the small intestine. This is major surgery with risks such as bleeding, infections and reactions to the anesthesia. Possible rare, but serious risks specific to RYGB include: death, blood clots in the legs, leaking at one of the staple lines in the stomach, pneumonia, narrowing of the opening between the stomach and the small intestines and dumping syndrome. Possible complication of gastric bypass surgery include: vitamin and mineral deficiency, dehydration, gallstones, bleeding stomach ulcer, hernia at the incision site, intolerance to certain foods, kidney stones and low blood sugar (hypoglycemia).

Biliopancreatic diversion with a duodenal switch (BPD-DS), usually referred to as a “duodenal switch,” is a complex bariatric operation that principally includes 1) removing a large portion of the stomach to promote smaller meal sizes, 2) re-routing of food away from much of the small intestine to partially prevent absorption of food, and 3) re-routing of bile and other digestive juices which impair digestion. In removing a large portion of the stomach, a more tubular “gastric sleeve”, also known as a vertical sleeve gastrectomy, or VSG (see below) is created. The smaller stomach sleeve remains connected to a very short segment of the duodenum, which is then directly connected to a lower part of the small intestine. This operation leaves a small portion of the duodenum available for food and the absorption of some vitamins and minerals. However, food that is eaten by the patient bypasses the majority of the duodenum. The distance between the stomach and colon is made much shorter after this operation, thus promoting malabsorption. BPD-DS produces significant weight loss. However, there is greater risk of long-term complications because of decreased absorption of food, vitamins, and minerals.

Vertical sleeve gastrectomy (VSG) historically had been performed only as the first stage of BPD-DS (see above) in patients who may be at high risk for complications from more extensive types of surgery. These patients' high risk levels are due to body weight or medical conditions. However, more recent information indicates that some patients who undergo a VSG can actually lose significant weight with VSG alone and avoid a second procedure. It is not yet known how many patients who undergo VSG alone will need a second stage procedure. A VSG operation restricts food intake and does not lead to decreased absorption of food. However, most of the stomach is removed, which may decrease production of a hormone called ghrelin. A decreased amount of ghrelin may reduce hunger more than other purely restrictive operations, such as gastric band.

Adjustable gastric band (AGB) works primarily by decreasing food intake. Food intake is limited by placing a small bracelet-like band around the top of the stomach to produce a small pouch about the size of a thumb. The outlet size is controlled by a circular balloon inside the band that can be inflated or deflated with saline solution to meet the needs of the patient. Risks and complications include: bleeding, infection, slippage of the band can occur with persistent vomiting, the band may erode into the inside of the stomach, the band can spontaneously deflate due to leakage and the stomach pouch can enlarge, the stoma (stomach outlet) can be blocked, gastroesophageal reflux or GERD can occur and the risk or death due to gastric banding surgery is about one in 2,000.

### BRIEF DESCRIPTION OF THE FIGURES

FIG. 1 shows an example device of the present invention in an unwrapped, or open, conformation; the narrow portions at the top and bottom of the figure are configured to encompass the pyloric and lower esophageal sphincters; the wide portion between the narrow portions is configured to encompass the body of the stomach; dotted lines depict regions of the wrap that have been folded back to display portions of the fasteners located on the opposite side of the wrap.

FIG. 2 shows an example device of the present invention in a wrapped, or closed, conformation.

### SUMMARY OF THE INVENTION

The invention relates to devices, systems and methods for weight reduction. Specifically it relates to the reduction of the stomach with the use of an adjustable wrap around the stomach (e.g., the entire stomach) that is less invasive compared to other stomach reduction surgeries.

The major problem with using any device that is wrapped around the stomach is dealing with the formation of adhesions to the intra-abdominal viscera or organs contained within the abdominal cavity; they include the stomach, intestines, liver, spleen, pancreas, and parts of the urinary and reproductive tracts. In an attempt to minimize or even eliminate such formations, the present invention provides a barrier system to prevent contact between the viscera organs and a biomaterial needs to be used.

For example, in some embodiments of the present invention, a polypropylene mesh coated (e.g., coated on both sides) with 2-hydroxyethyl methacrylate (p(HEMA)) hydrogel is used to avoid these adhesions. It will be understood that a variety of other meshes or materials and/or coating may be employed. The above examples are provided simply to illustrate aspects of the invention. With the progress of materials sciences, tissue engineering, and multidisciplinary approaches, it has been possible to design and characterize a number of different materials for specific medical needs. The hydrogels belong to a class of materials that can be made through several methods, which may determine the properties of the product and direct its applications. They are defined as tridimensional nets made of polymeric chains that swell but do not dissolve. Hydrogel is originally a copolymer of etileno glycol dimethacrylate developed by Wichterle and Lim in 1954, and was the base used to make the first soft contact lenses. The synthetic polymers represented by hydrogels are made of flexible and light hydrophilic resin and have been used for the production of several devices. The hydrogel of 2-hydroxyethyl methacrylate (p(HEMA)) is a light, flexible, biocompatible, and non-toxic material which presents no antigenic activity. The properties of 2-hydroxyethyl methacrylate (p(HEMA)) hydrogel do not induce necrosis, tumors or infections but, rather, it is well tolerated, non-toxic and biocompatible.

Polypropylene mesh has been the most used biomaterial due to its resiliency, easy to manipulate and inexpensive. The disadvantage of the polypropylene mesh-p(HEMA) composite is its stiffness, but the quality or state of being malleable by virtue of which the material can be extended in all directions without rupture by the application makes it a suitable material for this specific medical need.

In embodiments of the present invention, with the polypropylene mesh coated on both sides with 2-hydroxyethyl methacrylate (p(HEMA)) hydrogel that is wrapped around, overlapping and covering the or a portion of the stomach beginning with the lower esophageal sphincter and ending with the pyloric sphincter attached with fasteners. The center or remaining body of the stomach will have adjustable fasteners. The invention is not limited to the type of materials used for, or the nature of, the fasteners.

Since this procedure does not involve an intestinal bypass, laparoscopic adjustable gastric wrapping (LAGW) is a procedure which induces weight loss solely through the restriction of food intake. For optimal results, strict patient compliance and follow-up for wrap adjustments are required. The adjustable gastric wrap (AGW) is a reversible procedure that does not carry the risks of nutritional and mineral deficiencies or many of the other negative side effects from other bariatric procedures.

This present invention is different from the adjustable gastric band (AGB) in that the band produces a small pouch about the size of a thumb resulting in risks and complications that include: bleeding, infection, slippage of the band can occur with persistent vomiting, the band may erode into the inside of the stomach, the band can spontaneously deflate due to leakage and the stomach pouch can enlarge, the stoma (stomach outlet) can be blocked, gastroesophageal reflux or GERD can occur and the risk or death due to gastric banding surgery is about one in 2,000. After the lap band surgery is performed, most patients must dramatically alter their eating habits. Patients must stick to a liquid diet the first one to two weeks after the surgery, and must consume pureed foods in the week or two following that. Foods with high calorie, fat and sugar content, such as pastries, popcorn, pastas and fried foods, typically need to be avoided indefinitely after the surgery. Because the surgery reduces the amount of food the stomach can hold, meals must be small and measured or the patient may experience vomiting or diarrhea. Some experience depression and self-esteem issues if the weight loss has left them with extra skin due to a rapid weight lose.

The present invention utilizes the whole body of the stomach or a substantial portion thereof with the surgeon making periodic adjustments based on weight loss, food cravings and other physical reactions to the surgery. The adjustments are not as dramatic as with (AGB), reducing most if not all the possible side effects associated with (AGB).

The placement and adjustment of the device can be done with standard laparoscopic technique which uses up to six ports in various configurations to safely accomplish the procedure and or with single-incision laparoscopic surgery (SILS) for placement and adjusting the adjustable gastric wrap (AGW). (SILS) upholds the principal advantages of minimal access surgery including shortened hospital stays, the potential for decreased postoperative pain, and cosmetically acceptable scars by reducing large or multiple incisions to a relatively small, single one. A liver retractor technique eliminated one of the typical incisions utilized in other described “single-incision” techniques.

The present invention is not to be limited by the properties of the 2-hydroxyethyl methacrylate (p(HEMA)) hydrogel; shape, size or composition of mesh used; shape, size, type of fasteners adjustment wise or not; any additional aspects of this invention will be apparent to persons skilled in the relevant art based on the teachings contained herein.

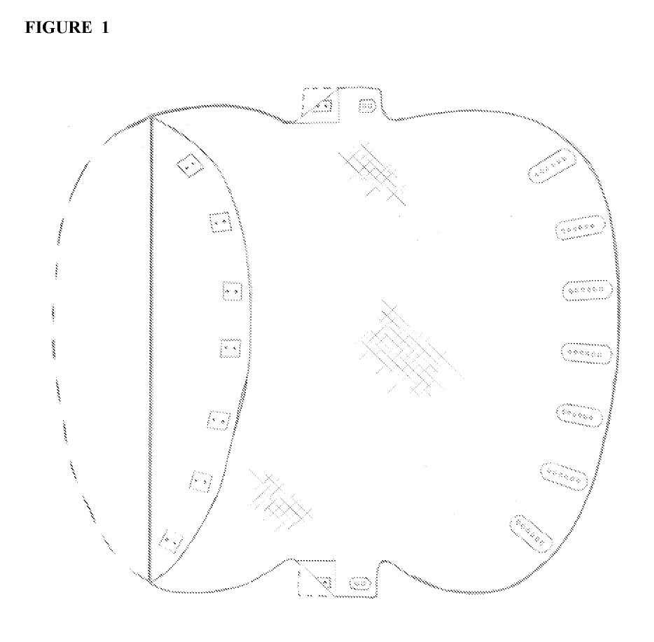

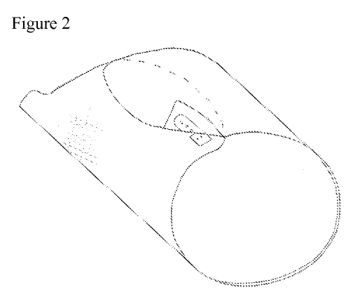

FIGS. 1 and 2 show exemplary embodiments of the device.

I claim:

1. A method for inducing weight reduction in a subject, comprising: a) applying a restrictive wrap around the stomach of said subject so as to at least substantially cover the outer surface of the stomach; and b) tightening the wrap to restrict the size of the stomach.

2. The method of claim 1, wherein said restrictive wrap comprises a polypropylene mesh material.

3. The method of claim 2, wherein said polypropylene mesh material is coated with 2-hydroxyethyl methacrylate hydrogel.

4. The method of claim 4, wherein said polypropylene mesh material is coated on both sides with 2-hydroxyethyl methacrylate hydrogel.

5. The method of claim 1, wherein said restrictive wrap comprises adjustable fasteners that allow for said tightening step.

6. The method of claim 1, wherein said applying and tightening steps are performed laparoscopically.

7. The method of claim 1, wherein said restrictive wrap substantially cover the outer surface of the stomach from the lower esophageal sphincter to the pyloric sphinter.

8. The method of claim 1, wherein tightening the wrap restricts the amount of food that can be taken into the stomach.

9. The method of claim 1, wherein said tightening step is repeated one or more times to further restrict the volume of said stomach multiple tightening steps.

10. The method of claim 1, wherein said repeating is performed over the course of 1 week to 10 years.

11. A restrictive stomach wrap comprising:
(a) first and second narrow portions, wherein said narrow portions comprise fasteners configured to secure said narrow portions around a tubularly shaped object, said first narrow portion being configured to encompass the exterior of the lower esophageal sphincter, said second narrow portion being configured to encompass the exterior of the pyloric sphincter; and
(b) a wide portion, relative to said first and second narrow portions, located between said first and second narrow portions, comprising adjustable fasteners configured to secure said wide portion around an object of varying widths, said wide portion being configured to substantially encompass said stomach.

12. The restrictive stomach wrap of claim 11, wherein said restrictive stomach wrap comprises a polypropylene mesh material.

13. The restrictive stomach wrap of claim 12, wherein said polypropylene mesh material is coated with 2-hydroxyethyl methacrylate hydrogel.

14. The restrictive stomach wrap of claim 11, wherein said polypropylene mesh material is coated on both sides with 2-hydroxyethyl methacrylate hydrogel.

15. The restrictive stomach wrap of claim 13, wherein said restrictive stomach wrap is coated with 2-hydroxyethyl methacrylate hydrogel.

16. The restrictive stomach wrap of claim 14, wherein said restrictive stomach wrap is coated with 2-hydroxyethyl methacrylate hydrogel.
